(19) United States
(12) Patent Application Publication
(10) Pub. No.: US 2018/0168550 A1
(43) Pub. Date: Jun. 21, 2018

(54) ULTRASOUND IMAGING APPARATUS AND METHOD OF CONTROLLING THE SAME

(71) Applicant: SAMSUNG MEDISON CO., LTD., Hongcheon-gun (KR)

(72) Inventors: Won Ik HEO, Goyang-si (KR); Eun Ho YANG, Seoul (KR); Ae Ri YOUN, Seoul (KR); Jin JEON, Seongnam-si (KR)

(21) Appl. No.: 15/461,234

(22) Filed: Mar. 16, 2017

(30) Foreign Application Priority Data
Dec. 19, 2016 (KR) 10-2016-0173650

(51) Int. Cl. A61B 8/00; A61B 8/08; A61B 8/14

(52) U.S. Cl. CPC A61B 8/4477; A61B 8/461; A61B 8/54; A61B 8/14; A61B 8/08; A61B 8/0825; A61B 8/467

(57) ABSTRACT

It is an aspect of the present disclosure to provide an ultrasound imaging apparatus capable of recommending a probe adequate for an organ of a patient to be diagnosed to allow a user to replace an existing probe to efficiently and precisely diagnose the patient and a method of controlling the same.

In accordance with one aspect of the present disclosure, an ultrasound imaging apparatus comprising: a first ultrasound probe configured to obtain an ultrasonic image of an object by transmitting and receiving ultrasonic signals; a second ultrasound probe of a different type from the first ultrasound probe; a display portion configured to display an ultrasonic image obtained by at least one of the first ultrasound probe and the second ultrasound probe; and a controller configured to derive information of the object based on an ultrasonic image obtained by the first ultrasound probe and guide replacing the first ultrasound probe with the second ultrasound probe based on the information of the object.

### CROSS-REFERENCE TO RELATED APPLICATION

This application claims the benefit of Korean Patent Application No. 10-2016-0173650, filed on Dec. 19, 2016 in the Korean Intellectual Property Office, the disclosure of which is incorporated herein by reference.

### BACKGROUND

### 1. Field

Embodiments of the present disclosure relate to an ultrasound imaging apparatus configured to generate an internal image of an object using ultrasonic waves.

### 2. Description of the Related Art

An ultrasound imaging apparatus is an apparatus that emits ultrasonic signals at a body surface of an object toward a target part inside the body and noninvasively obtains an image of a cross section of soft tissue or a blood flow using information of reflected ultrasonic signals (ultrasonic echo signals).

Compared with other image diagnosis apparatuses such as an X-ray diagnosis apparatus, an X-ray computerized tomography (CT) scanner, an magnetic resonance image (MRI), a nuclear medicine diagnosis apparatus and the like, since the ultrasound imaging apparatus has a small size, is at a low price, is able to display in real time, and is free from radiation exposure with high safety, the ultrasound imaging apparatus is generally used for cardiac, abdominal, urinary, and ob-gyn diagnoses.

The ultrasound imaging apparatus includes an ultrasound probe configured to transmit ultrasonic signals and receive ultrasonic echo signals reflected from the object to an object to obtain an ultrasonic image of the object and a main body configured to generate an internal image of the object using the ultrasonic echo signals received at the ultrasound probe.

### SUMMARY

It is an aspect of the present disclosure to provide an ultrasound imaging apparatus capable of recommending a probe adequate for an organ of a patient to be diagnosed to allow a user to replace an existing probe to efficiently and precisely diagnose the patient and a method of controlling the same.

Additional aspects of the present disclosure will be set forth in part in the description which follows and, in part, will be obvious from the description, or may be learned by practice of the present disclosure.

In accordance with one aspect of the present disclosure, an ultrasound imaging apparatus comprising: a first ultrasound probe configured to obtain an ultrasonic image of an object by transmitting and receiving ultrasonic signals; a second ultrasound probe of a different type from the first ultrasound probe; a display portion configured to display an ultrasonic image obtained by at least one of the first ultrasound probe and the second ultrasound probe; and a controller configured to derive information of the object based on an ultrasonic image obtained by the first ultrasound probe and guide replacing the first ultrasound probe with the second ultrasound probe based on the information of the object.

The ultrasound imaging apparatus further comprises: an input portion configured to receive information related to the ultrasonic image input from a user,

wherein the controller guides to replace the first ultrasound probe with the second ultrasound probe based on the information received by the input portion.

The ultrasound imaging apparatus further comprises: an input portion configured to receive information of the object input from a user,

The controller may guide to replace the first ultrasound probe with the second ultrasound probe based on the information received by the input portion.

The controller may store an ultrasonic image obtained by the first ultrasound probe and displays the ultrasonic image obtained by the first ultrasound probe and an ultrasonic image obtained by the second ultrasound probe on the display portion when the first ultrasound probe is replaced with the second ultrasound probe.

The controller may display a moving image for guiding the second ultrasound probe to the same position as that of the first ultrasound probe on the display portion when the first ultrasound probe is replaced with the second ultrasound probe.

Each of the first ultrasound probe and the second ultrasound probe may be provided as any one of a convex array probe, a linear array probe, an endocavity array probe, and a phased array probe.

The second ultrasound probe may obtain an ultrasonic image using ultrasonic waves at a different frequency band from that of the first ultrasound probe.

The controller may guide replacing the first ultrasound probe with the second ultrasound probe that is a linear array probe when the object is determined as a gallbladder.

The controller may guide replacing the first ultrasound probe with the second ultrasound probe that is a linear array probe when the object is determined as a liver.

The controller may guide replacing the first ultrasound probe with the second ultrasound probe that is an endocavity array probe when the object is determined as a uterus.

The controller may display information of the second ultrasound probe on the display portion when it is guided to replace the first ultrasound probe with the second ultrasound probe.

In accordance with one aspect of the present disclosure, a method of controlling an ultrasound imaging apparatus, comprising: obtaining an ultrasonic image of an object by transmitting and receiving ultrasonic signals; deriving information of the object based on an ultrasonic image obtained by a first ultrasound probe; guiding replacing the first ultrasound probe with a second ultrasound probe based on the information of the object; and displaying an ultrasonic image obtained by at least one of the first ultrasound probe and the second ultrasound probe.

The guiding of replacing the first ultrasound probe with the second ultrasound probe may comprise: receiving information related to the ultrasonic image input from a user; and guiding an input portion to replace the first ultrasound probe with the second ultrasound probe based on the information received at the input portion.

The guiding of replacing the first ultrasound probe with the second ultrasound probe may comprise: receiving information of the object input from a user; and guiding an input portion to replace the first ultrasound probe with the second ultrasound probe based on the information received at the input portion.

The method of controlling an ultrasound imaging apparatus further comprising storing the ultrasonic image obtained by the first ultrasound probe, wherein the displaying of the ultrasonic image comprises displaying the ultrasonic image obtained by the first ultrasound probe and an ultrasonic image obtained by the second ultrasound probe when the first ultrasound probe is replaced with the second ultrasound probe.

The guiding of replacing the first ultrasound probe with the second ultrasound probe may comprise displaying a moving image for guiding the second ultrasound probe to the same position as that of the first ultrasound probe.

The guiding of replacing the first ultrasound probe with the second ultrasound probe may comprise guiding replacing the first ultrasound probe with the second ultrasound probe that is a linear array probe when the object is determined as a gallbladder.

The guiding of replacing the first ultrasound probe with the second ultrasound probe may comprise guiding replacing the first ultrasound probe with the second ultrasound probe that is a linear array probe when the object is determined as a liver.

The guiding of replacing the first ultrasound probe with the second ultrasound probe may comprise guiding replacing the first ultrasound probe with the second ultrasound probe that is an endocavity array probe when the object is determined as a uterus.

The guiding of replacing the first ultrasound probe with the second ultrasound probe may comprise displaying information of the second ultrasound probe when it is guided that the first ultrasound probe is replaced with the second ultrasound probe.

### BRIEF DESCRIPTION OF THE DRAWINGS

These and/or other aspects of the present disclosure will become apparent and more readily appreciated from the following description of the embodiments, taken in conjunction with the accompanying drawings of which:

FIG. 1 is an external view of an ultrasound imaging apparatus in accordance with one embodiment.

FIG. 2 is a control block diagram of the ultrasound imaging apparatus in accordance with one embodiment.

FIG. 3 is a control block diagram illustrating a configuration of a main body of the ultrasound imaging apparatus in accordance with one embodiment in detail.

FIG. 4 is a schematic control block diagram illustrating the configuration of the main body of the ultrasound imaging apparatus according to one embodiment.

FIG. 5 is a view illustrating ultrasound probes according to one embodiment.

FIGS. 6A and 6B are views illustrating ultrasonic images of a gallbladder taken with different types of ultrasound probes.

FIGS. 7A and 7B are views illustrating ultrasonic images of a liver taken with different types of ultrasound probes.

FIGS. 8A and 8B are views illustrating ultrasonic images of a uterus taken with different types of ultrasound probes.

FIGS. 9A and 9B illustrate ultrasonic images of breasts taken using a probe with different frequencies.

FIG. 10 illustrates an image of a gallbladder taken using different types of probes on one display according to one embodiment.

FIG. 11 illustrates adjusting positions of images of a gallbladder taken using different types of probes to the same position in accordance with one embodiment.

FIG. 12 illustrates changes in images shown on a display in accordance with one embodiment.

FIGS. 13 to 15 are flowcharts according to one embodiment.

### DETAILED DESCRIPTION

Hereinafter, exemplary embodiments of the present invention will be described in greater detail with reference to the appended drawings.

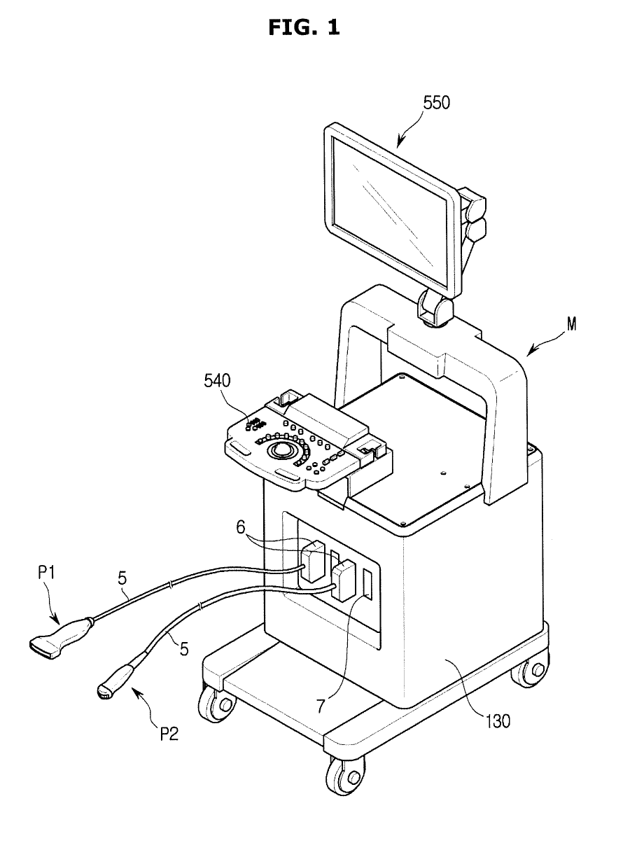

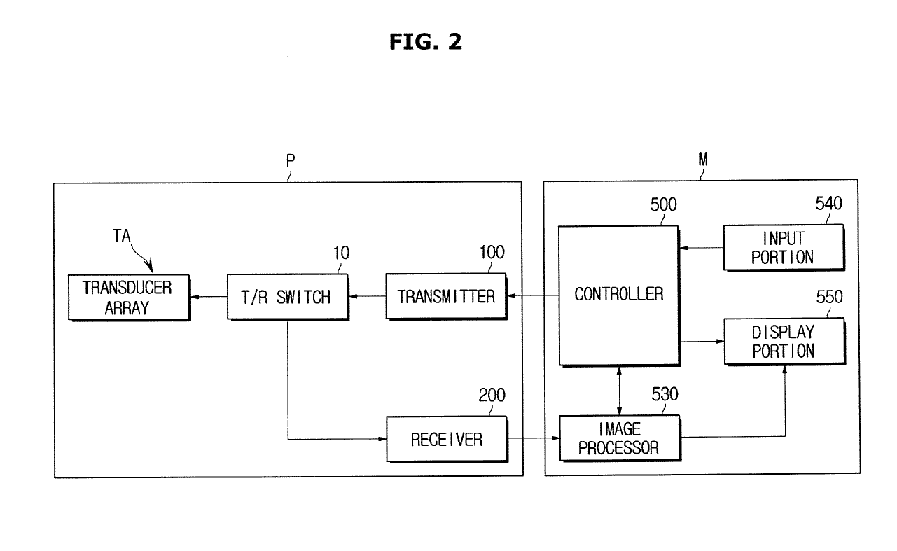

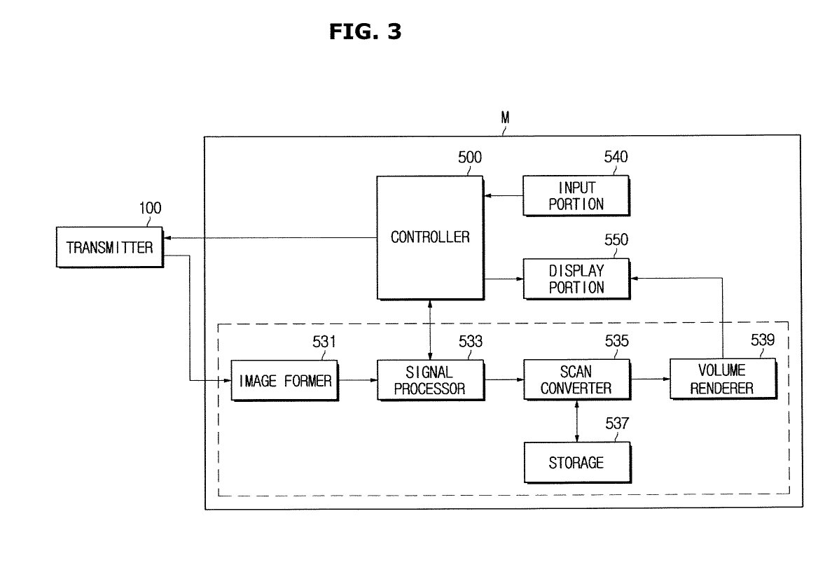

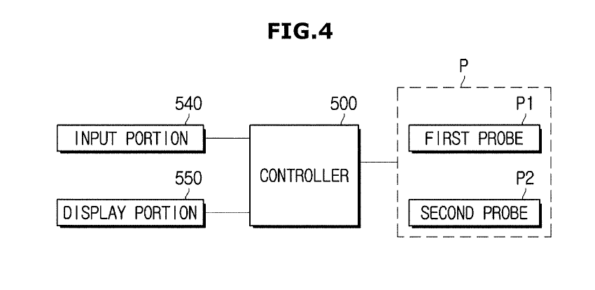

FIG. 1 is an external view of an ultrasound imaging apparatus in accordance with one embodiment, and FIG. 2 is a control block diagram of the ultrasound imaging apparatus in accordance with one embodiment. Also, FIG. 3 is a control block diagram illustrating a configuration of a main body of the ultrasound imaging apparatus in accordance with one embodiment in detail. Also, FIG. 4 is a schematic control block diagram illustrating the configuration of the main body of the ultrasound imaging apparatus according to one embodiment.

Referring to FIG. 1, an ultrasound imaging apparatus 1 includes an ultrasound probe P configured to transmit ultrasonic waves to an object, receive ultrasonic echo signals from the object, and convert the ultrasonic echo signals into electrical signals and a main body M connected to the ultrasound probe P and configured to include an input portion 540 and a display portion 550 and display an ultrasonic image. The ultrasound probe P may be connected to the main body M of the ultrasound imaging apparatus 1 through a cable 5, may receive various signals necessary for controlling the ultrasound probe P, and may transfer analog signals or digital signals corresponding to the ultrasonic echo signals received by the ultrasound probe P to the main body M. However, embodiments of the ultrasound probe P are not limited thereto and the ultrasound probe P may be embodied as a wireless probe to transmit and receive signals through a network formed between the ultrasound probe P and the main body M.

One end of the cable 5 may be connected to the ultrasound probe P and the other end thereof may include a connector 6 configured to be couplable with or separable from a slot 7 of the main body M. The main body M and the ultrasound probe P may send and receive control commands or data through the cable 5. For example, when a user inputs information with respect to a focal depth, a size or shape of an aperture, a steering angle or the like through the input portion 540, such pieces of information may be transferred to the ultrasound probe P through the cable 5 to be used for forming beams transmitted and received between a transmitter 100 and a receiver 200. Otherwise, when the ultrasound probe P is embodied as a wireless probe as described above, the ultrasound probe P is connected to the main body M through a wireless network not the cable 5. When connected to the main body M through a wireless network, the main body M and the ultrasound probe P may send and receive the control commands or data described above. The main body M may include a controller 500, an image processor 530, the input portion 540, and the display portion 550 as shown in FIG. 2.

The controller 500 controls overall operations of the ultrasound imaging apparatus 1. In detail, the controller 500 generates control signals for controlling each of components of the ultrasound imaging apparatus 1 such as the transmitter 100, a T/R switch 10, the receiver 200, the image processor 530, and the display portion 550 as shown in FIG. 2 and controls operations of all the components described above. In the ultrasound imaging apparatus 1 according to the embodiment shown in FIGS. 2 and 3, a transmission/reception beamformer is included in the ultrasound probe P not the main body M. However, the transmission/reception beamformer may be included in the main body M not the ultrasound probe P.

The controller 500 calculates a delay profile of a plurality of ultrasonic transducer elements 60 that form an ultrasonic transducer array TA and calculates a time delay value according to a distance difference of a focal point between the object and each of the plurality of ultrasonic transducer elements 60 included in the ultrasonic transducer array TA based on the calculated delay profile. Also, the controller 500 controls the transmitter/received beamformer according thereto to generate transmission/reception signals.

Also, the controller 500 may control the ultrasound imaging apparatus 1 by generating a control command for each component of the ultrasound imaging apparatus 1 according to an instruction or command of the user input through the input portion 540. The controller 500 according to the disclosed embodiment sets a region of interest (ROI) in an ultrasonic image, particularly, in a sonoelastographic image displayed on the display portion 550 as a shape corresponding to that of an interested object instead of a simple circular or quadrangular shape including the interested object. For example, in a sonoelastographic image of a uterine cervix, a shape corresponding to a shape of the uterine cervix that is an object of interest is set as an ROI and is displayed in the sonoelastographic image displayed on a display portion. A detailed description thereof will be described below.

The image processor 530 generates an ultrasonic image of a target part inside the object based on ultrasonic signals focused by the receiver 200.

Referring to FIG. 3, the image processor 530 may include an image former 531, a signal processor 533, a scan converter 535, a storage 537, and a volume renderer 539.

The image former 531 generates a two-dimensional or three dimensional coherent image of a target part inside an object based on ultrasonic signals collected by the receiver 200.

The signal processor 533 converts coherent image information formed by the image former 531 into ultrasonic image information according to a diagnosis mode such as a brightness mode (B-mode), a Doppler mode (D-mode) or the like. For example, when a diagnosis mode is set as a B-mode, the signal processor 533 performs processing such as an A/D conversion treatment and compiles ultrasonic image information for a B-mode image in real time. Also, when a diagnosis mode is set as a D-mode, the signal processor 533 extracts phase shift information from an ultrasonic signal, calculates information such as a blood current corresponding to each point of a photographed cross section such as a speed, power, and dispersion, and compiles ultrasonic image information of the D-mode image in real time.

The scan converter 535 converts converted ultrasonic image information input from the signal processor 533 or converted ultrasonic image information stored in the storage 537 into a general video signal for the display portion 550 and transmits the video signal to the volume renderer 539.

The storage 537 temporarily or non-temporarily stores the ultrasonic image information converted through the signal processor 533.

The volume renderer 539 performs volume rendering based on the video signal transmitted from the scan converter 535, generates a final result image by correcting rendered image information, and transmits the generated result image to the display portion 550.

The input portion 540 may be provided to allow the user to input commands related to operations of the ultrasound imaging apparatus 1. The user may input or set diagnosis mode selection commands such as an ultrasonic diagnosis starting command, a B-mode, a motion mode (M-mode), a D-mode, an elastic mode, a three-dimensional mode and the like and ROI setting information including a size and a position of an ROI.

The B-mode is displaying an internal cross-sectional image of an object and illustrates a part with a strong reflected echo and a part with a weak reflected echo using a difference in brightness. A B-mode image is configured based on information obtained from several tens to several hundreds of scan lines.

The M-mode is displaying how biometric information (for example, brightness information) with respect to a certain part (M line) among cross-sectional images (B-mode images) of the object changes according to time as an image. Generally, the B-mode image and the M-mode image are displayed in one screen at the same time to allow the user to make an accurate diagnosis by comparing and analyzing two data.

The D-mode means an image using the Doppler effect in which a frequency of a sound emitted from a moving object causes a change. The mode using the Doppler effect described above may be subdivided again into a power Doppler imaging (PDI) mode, a color flow mode (S flow), and a DPDI mode.

The PDI mode is displaying the degree of a Doppler signal or the number of a structure (red blood cells in blood) as an image and is less sensitive to an incident angle in such a way that there is no fake signal and an image is less attenuated. Also, since the PDI mode records reflected Doppler energy, the PDI mode is very sensitive to even detect a small blood vessel and a blood flow at a low speed.

The color flow (S flow) mode provides a PDI that shows power of a Doppler signal in a two-dimensional distribution and a velocity image that indicates a velocity of a Doppler signal in a two-dimensional distribution. A color flow mode image may not only visualize a blood flow in real time but also show a widespread blood flow state from a high velocity blood flow in a large blood vessel to a low velocity blood flow in a small blood vessel.

The DPDI mode means a directional image that shows directional information of a Doppler signal in the PDI mode in a two-dimensional distribution. Accordingly, there is an effect of more accurately detecting information with respect to a blood flow than the PDI. Also, an M-mode image may be generated with respect to a Doppler mode image.

The elastic mode means a method of obtaining a sonoelastographic image of an object using elastography. Here, the elastography indicates analyzing that a difference in metamorphic grades of tissue according to pressure is decreased because a harder structure such as a malignant mass has lower tissue elasticity. The sonoelastographic image means an image that quantitatively displays stiffness of tissue as described above. Particularly, the sonoelastography is generally used for inspections for a uterine cervix, a breast cancer, a prostate cancer or the like.

The three-dimensional mode generally means an image that displays a geometric three-dimensional structure or space including X, Y, and Z values that represent a depth, area, and height and may mean a series of images that are three-dimensional shapes and mean a three-dimensional effect or stereo effect. As one example, the user may display a shape of an embryonic face to show the embryonic face to parents using a stereo effect of a three-dimensional mode.

The input portion 540 may include various devices that allow the user to input an instruction or command such as a keyboard, a mouse, a trackball, a tablet, a touch screen module and the like.

The display portion 550 displays a menu or guidance necessary for echography and an ultrasonic image obtained during an echography process. The display portion 550 displays an ultrasonic image of a target part inside an object generated by the image processor 530. An ultrasonic image displayed on the display portion 550 may be a B-mode ultrasonic image, an elastic mode ultrasonic image, and a three-dimensional ultrasonic image. The display portion 550 may display various ultrasonic images according to the modes described above. When displaying a sonoelastographic image, the display portion 550 may display a color preset according to a grade of elasticity (that is, a shear modulus) of each point of an ROI. For example, the display portion 550 may display a point at a low elasticity grade such as a tumor in red and may display a point at a high elasticity grade in blue in the sonoelastographic image. The preset colors are not limited to red and blue and may be set variously according to the user's settings. Also, the display portion 550 may display the elasticity grade of each point in the ROI that is digitized. Also, the display portion 550, as described below, may display images obtained by a first ultrasound probe and a second ultrasound probe and may display a moving image for guiding an ultrasound probe.

The display portion 550 may be embodied in various well-known display devices such as a cathode ray tube (CRT), a liquid crystal display (LCD) and the like.

The ultrasound probe P according to one embodiment, as shown in FIG. 2, may include the ultrasonic transducer array TA, the T/R switch 10, the transmitter 100, and the receiver 200. The ultrasonic transducer array TA may be provided at an end portion of the ultrasound probe P. The ultrasonic transducer array TA means arranging of a plurality of ultrasonic transducer elements 60 in a one-dimensional or two-dimensional array. The ultrasonic transducer array TA generates ultrasonic waves while vibrating due to an applied pulse signal or an alternating current. The generated ultrasonic waves are transmitted to a target part inside an object. In this case, the ultrasonic waves generated at the ultrasonic transducer array TA may be transmitted with a plurality of target parts inside the object as focuses. In other words, the generated ultrasonic waves may be transmitted to the plurality of target parts while multi-focused.

The ultrasonic waves generated at the ultrasonic transducer array TA are reflected by the target parts inside the object and return to the ultrasonic transducer array TA. The ultrasonic transducer array TA receives ultrasonic echo signals that are reflected by the target parts and return. When the ultrasonic echo signals arrive, the ultrasonic transducer array TA vibrates at a certain frequency corresponding to a frequency of the ultrasonic echo signals and outputs an alternating current at a frequency corresponding to a vibration frequency. Accordingly, the ultrasonic transducer array TA comes to convert the received ultrasonic echo signals into certain electric signals. Since each of the ultrasonic transducer elements 60 receives an ultrasonic echo signal and outputs an electrical signal, the ultrasonic transducer array TA may output electrical signals in a plurality of channels.

An ultrasonic transducer may be embodied as any one of a magnetostrictive ultrasonic transducer using a magnetostrictive effect of a magnetic body, a piezoelectric ultrasonic transducer using a piezoelectric effect of a piezoelectric material, and a capacitive micromachined ultrasonic transducer (CMUT) that transmits and receives ultrasonic waves using vibrations of several hundreds or several thousands of micromachined thin films. Also, in addition thereto, other types of transducers capable of generating ultrasonic waves or generating electrical signals according to ultrasonic waves may also be examples of the ultrasonic transducer.

For example, the ultrasonic transducer elements 60 according to the disclosed embodiment may each include a piezoelectric vibrator or a thin film. When an alternating current is applied from a power source, the piezoelectric vibrator or the thin film vibrates at a certain frequency according to the alternating current and generates ultrasonic waves at a certain frequency according to the vibration frequency. On the other hand, when an ultrasonic echo signal at a certain frequency arrives at the piezoelectric vibrator or the thin film, the piezoelectric vibrator or the thin film vibrates according to the ultrasonic echo signal and outputs an alternating current at a frequency corresponding to the vibration frequency.

The transmitter 100 applies a transmission pulse to the transducer array TA to allow the transducer array TA to transmit an ultrasonic signal to a target part inside an object. The transmitter 100 may include a transmission beamformer 110 and a pulser 120.

The transmission beamformer 110 forms a transmission signal pattern according to a control signal of the controller 500 of the main body M and outputs the transmission signal pattern to the pulser 120. The transmission beamformer 110 forms the transmission signal pattern based on a time delay value with respect to each of the ultrasonic transducer elements 60 that form the ultrasonic transducer array TA calculated through the controller 500 and transmits the formed transmission signal pattern to the pulser 120.

The receiver 200 performs a certain process with respect to the ultrasonic echo signal received from the ultrasonic transducer array TA and performs reception beamforming. The receiver 200 may include a reception signal processor and a reception beamformer. An electrical signal converted by the ultrasonic transducer array TA is input to the reception signal processor. The reception signal processor may amplify a signal before signal-processing or time delay processing with respect to the electrical signal obtained by converting the ultrasonic echo signal and may adjust a gain or compensate attenuation according to a depth. In more detail, the reception signal processor may include a low noise amplifier (LNA) that decreases noise with respect to the electrical signal input from the ultrasonic transducer array TA and a variable gain amplifier (VGA) that controls a gain value according to the input signal. The VGA may perform time gain compensation (TGC) for compensating a gain according to a distance from a focusing point but is not limited thereto.

The reception beamformer performs beamforming with respect to the electrical signal input from the reception signal processor. The reception beamformer increases signal strength of the electrical signal input from the reception signal processor through superposition. The signal beamformed by the reception beamformer is converted into a digital signal while passing through an analog-to-digital converter and is transmitted to the image processor 530 of the main body M. When the analog-to-digital converter is provided at the main body M, an analog signal beamformed by the reception beamformer may be transmitted to the main body M and may be converted into a digital signal at the main body M. Otherwise, the reception beamformer may be a digital beamformer. The digital beamformer may include a storage capable of sampling and storing an analog signal, a sampling period controller capable of controlling a sampling period, an amplifier capable of adjusting amplitude of a sample, an anti-aliasing low pass filter for preventing aliasing before sampling, a bandpass filter capable of selecting a desired frequency band, an interpolation filter capable of increasing a sampling rate of beamforming, and a high-pass filter capable of removing a direct current (DC) component or a signal in a low frequency band.

Referring to FIG. 4, FIG. 4 is a schematic diagram obtained by simplifying the control block diagrams of FIGS. 2 and 3.

The input portion 540 may receive information of an object from a user. While diagnosing a patient, the user may receive a target of diagnosis of the patient and may input the name of a disease to be diagnosed through the input portion 540. Also, when to measure a length, width, or volume of an object, the user may input information related thereto into the input portion 540. Also, as described below, the controller 500 may guide the user to change a probe based thereon.

Also, the user may input information related to an ultrasonic image that is to be obtained, through the input portion 540. For example, the user may input an ROI of the object and information on a depth of the ultrasonic image. For example, when the ROI of the user is a boundary of a liver and diagnoses a patient with a convex array probe, since it is necessary to obtain an ultrasonic image with relatively high resolution, it is necessary to measure using a linear array probe. As described below, the controller 500 may guide the user to replace the convex array probe with the linear array probe.

A first ultrasound probe P1 and a second ultrasound probe P2 are ultrasound probes used by the user to diagnose a disease of the patient. The first ultrasound probe P1 and the second ultrasound probe P2 may be any types of ultrasound probes that will be described below. The user may diagnose a patient using the first ultrasound probe P1. When the user uses an inadequate probe as follows, the controller 500 may recommend replacing the probe with the second ultrasound probe P2. Detailed operations related thereto will be described below. Also, the first ultrasound probe P1 and the second ultrasound probe P2 are merely ordinal numbers for distinguishing ultrasound probes from one another and do not mean the number of probes or priority thereof. Also, the first ultrasound probe P1 and the second ultrasound probe P2 recommended by the controller 500 may be different types but may be the same type using different frequencies.

The controller 500 may recommend the most efficient ultrasound probe for the user to diagnose a disease of a patient. In recommendation of an ultrasound probe, the recommendation may be performed based on information related to an ultrasonic image and information of an object input by the user through the input portion 540. Also, a particular feature of each organ may be derived through the first ultrasound probe P1 and the organ may be recognized based on the derived feature to determine a type of the organ. That is, the controller 500 may derive information of an object based on an ultrasonic image obtained by the first ultrasound probe and may guide replacing an ultrasound probe based thereon. Also, the controller 500 may learn information with respect to an organ of a patient diagnosed by the user and may recommended the second ultrasound probe based on the information. As an example, the user may input a patient's gallbladder to be diagnosed into the input portion 540. When the user diagnoses the patient using the first ultrasound probe P1, the controller 500 may recommend the user to diagnose using the second ultrasound probe P2. In accordance with one embodiment of the present disclosure, when the first ultrasound probe P1 is replaced with the second ultrasound probe P2, the controller 500 may output information related to the second ultrasound probe P2 on the display portion 550. For example, a title of a type of the second ultrasound probe P2, an external shape of the second ultrasound probe P2, and identification information of the second ultrasound probe P2 previously input by the user may be output. Also, the controller 500 may control a moving image for guiding a position of the second ultrasound probe P2 to be displayed on the display portion 550. As an example, the moving image may be expressed as arrows in different sizes and colors. Types of ultrasound probes that may become the first ultrasound probe P1 and the second ultrasound probe P2 will be described below.

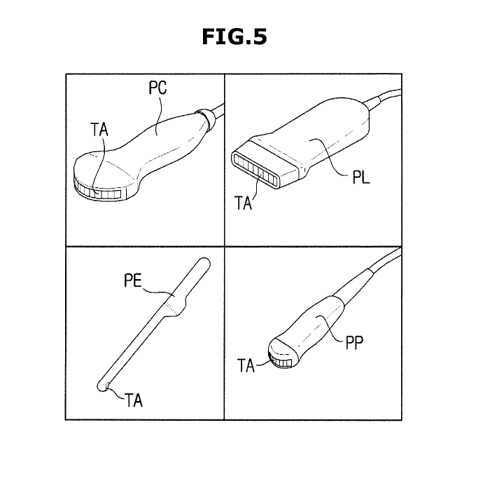

FIG. 5 is a view illustrating ultrasound probes according to one embodiment.

Ultrasound probes may be devices configured to generate and transmit ultrasonic waves and receive reflected echoes and may have different shapes and sizes depending on a target and purpose of inspection. A probe includes a matching layer that comes into contact with the surface of a human body, a backing layer that absorbs a back sound, and a piezoelectric material therebetween and may further include an insulator that prevents a leakage of ultrasonic waves, a cable and the like. The matching layer reduces a difference in acoustic resistance between the probe and the skin and allows the probe to efficiently transmit ultrasonic beams to the inside of tissue and to receive the reflected beams with high sensitivity. The backing layer may generally absorb a back sound and may include tungsten and rubber powder.

Probes may be classified into a linear array type, a convex array type, an endocavity convex array type, and a phased array type, depending on an array of a piezoelectric material that is a component material thereof.

A linear array probe PL is a probe in which elements of the probe are linearly arranged and may linearly transmit and receive ultrasonic waves. The linear array probe PL may be used for diagnosing a part near the skin and may obtain an image of high resolution. The linear array probe PL may be used for inspecting breasts, thyroid, muscles and skeletons, and a blood vascular system.

A convex array probe PC is a probe in which elements of the probe are curvedly arranged to curvedly transmit and receive ultrasonic waves and may be used for widely diagnosing a part deep inside body. Accordingly, the convex array probe PC may be generally used to diagnose the abdomen and in obstetrics and gynecology.

An endocavity array probe PE is a probe in which elements of the probe are curvedly arranged and curvedly transmits and receives ultrasonic waves and may be used to diagnose a part close to the probe while being inserted into a body cavity. The endocavity array probe PE may be used in obstetrics, gynecology and urology.

A phased array probe PP is a probe in which elements are linearly arranged to transmit and receive ultrasonic waves while adjusting angles of the elements. The phased array probe PP may transmit and receive ultrasonic waves between ribs to be used for diagnosing a heart.

The types of ultrasound probes shown in FIG. 5 are merely an example of types of ultrasound probes and the type of the ultrasound probe used in the embodiment of the present disclosure is not limited thereto.

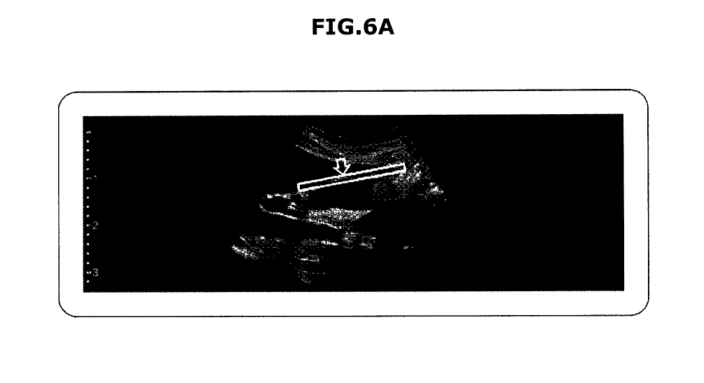

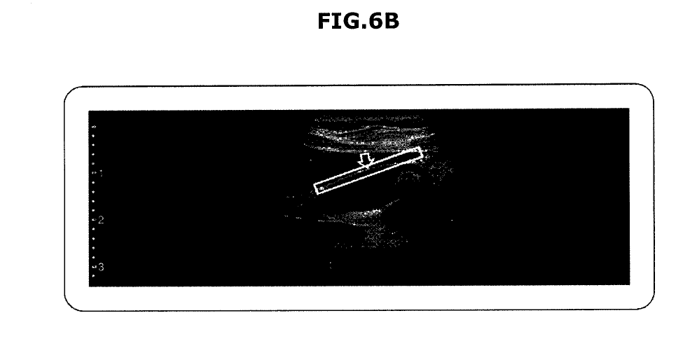

FIGS. 6A and 6B are views illustrating ultrasonic images of a gallbladder taken with different types of ultrasound probes.

Referring to FIGS. 6A and 6B, FIG. 6A illustrates an ultrasonic image of the gallbladder taken using the convex array probe PC and FIG. 6B illustrates an ultrasonic image of the gallbladder taken using the linear array probe PL. An ultrasound probe may be used to detect a gallbladder polyp and may use ultrasonic waves at a high frequency of 7.5 MHz to 12 MHz to obtain an ultrasonic image without interference of thick abdominal walls and gases inside intestinal canals. Also, an ultrasonic image with high resolution is necessary to diagnose the gallbladder.

In FIGS. 6A and 6B, since it is possible to compare the ultrasonic images taken using the convex array probe PC and the linear array probe PL, a difference in resolutions of wall parts of the gallbladder may be recognized. In detail, in the ultrasonic image taken using the convex array probe PC, the wall part is blurredly shown. On the other hand, in the ultrasonic image taken using the linear array probe PL, the wall of the gallbladder is clearly shown. Accordingly, when a polyp at the gallbladder is detected using ultrasonic images, the linear array probe PL is more effective than the convex array probe PC. Also, since the gallbladder is not positioned at a place relatively deep inside a human body, it is possible to take an image thereof using only the linear array probe PL. Accordingly, according to one embodiment of the present disclosure, when the user would like to obtain an ultrasonic image of gallbladder using another probe other than the linear array probe PL, the controller 500 may recommend the user to use the linear array probe PL. Through this, the user may use an ultrasound probe capable of obtaining a more precise image of the gallbladder.

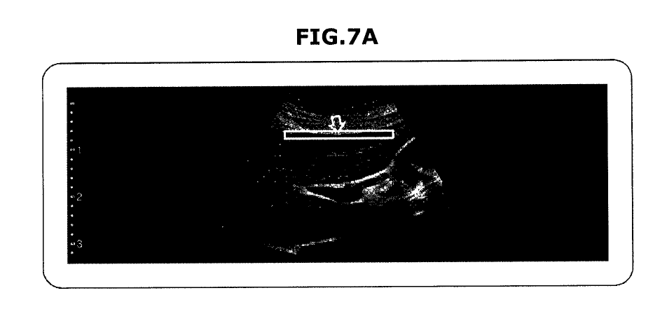

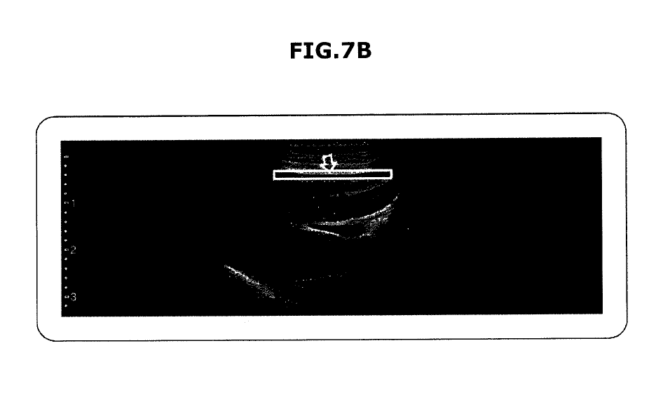

FIGS. 7A and 7B are views illustrating ultrasonic images of a liver taken with different types of ultrasound probes.

Referring to FIGS. 7A and 7B, FIG. 7A illustrates an ultrasonic image of the liver taken using the convex array probe PC and FIG. 7B illustrates an ultrasonic image of the liver taken using the linear array probe PL. An ultrasound probe may be used to diagnose cirrhosis, liver cancer, fatty liver and the like. Particularly, to diagnose the diseases described above, it is necessary to precisely check a boundary wall of the liver.

In FIGS. 7A and 7B, since it is possible to compare the ultrasonic images taken using the convex array probe PC and the linear array probe PL, a difference in resolutions of wall parts of the liver may be recognized. In detail, in the ultrasonic image taken using the convex array probe PC, a wall part is blurredly shown. On the other hand, in the ultrasonic image taken using the linear array probe PL, the wall of the liver is clearly shown. Accordingly, when a degree of liver cirrhosis is determined using ultrasonic images, the linear array probe PL is more effective than the convex array probe PC. Also, since the liver is not positioned at a place relatively deep inside a human body, it is possible to take an image thereof using only the linear array probe PL. Accordingly, according to one embodiment of the present disclosure, when the user would like to obtain an ultrasonic image of a liver using another probe other than the linear array probe PL, the controller 500 may recommend the user to use the linear array probe PL. Through this, the user may use a probe capable of obtaining a more precise image of the liver.

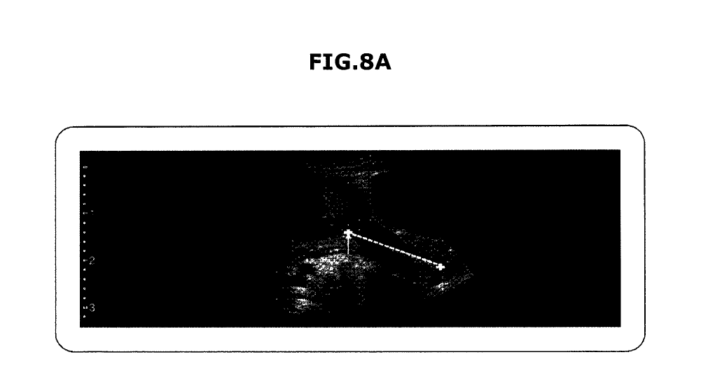

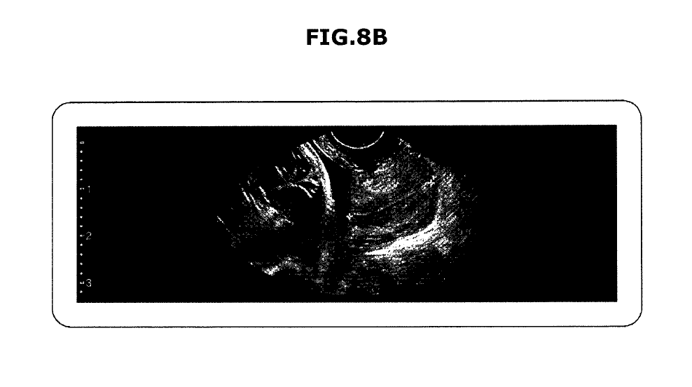

FIGS. 8A and 8B are views illustrating ultrasonic images of a uterus taken with different types of ultrasound probes.

Referring to FIGS. 8A and 8B, FIG. 8A illustrates an ultrasonic image of the uterus taken using the convex array probe PC and FIG. 8B illustrates an ultrasonic image of the uterus taken using the endocavity array probe PE. An ultrasound probe may be used for diagnosing and shadow-tracking pregnancy and a uterine myoma. Particularly, it is necessary that a probe is positioned close to the uterus to diagnose the diseases described above.

In FIGS. 8A and 8B, it is possible to compare ultrasonic images taken with the convex array probe PC and the endocavity array probe PE. In detail, in the ultrasonic image taken using the convex array probe PC, a wall part is blurredly shown. On the other hand, in the ultrasonic image taken using the endocavity array probe PE, since the resolution of the image is high, the image is clearly shown. Accordingly, when the uterus is diagnosed using ultrasonic images, since the endocavity array probe PE is capable of more precisely measuring a length of uterine cervix, the endocavity array probe PE is more effective than the convex array probe PC. However, a transabdominal ultrasound may be performed using the convex array probe PC or the linear array probe PL and performed while filling a bladder with urine to prevent obstruction of a visual field caused by fat, air, and the like inside a pelvic cavity. Compared with the endocavity array probe PE, it is possible to observe a broader part at the same time. Accordingly, according to one embodiment of the present disclosure, when the user would like to obtain an ultrasonic image of a uterus using another probe other than the endocavity array probe PE, the controller 500 may recommend the user to use the endocavity array probe PE. Through this, the user may use an ultrasound probe capable of obtaining a more precise image of the uterus.

In FIGS. 6A to 8B, although the types of the probes and body organs that are to be diagnosed have been described with respect to necessity of one embodiment of the present disclosure, recommended operations of an ultrasound probe for optimization are not limited thereto.

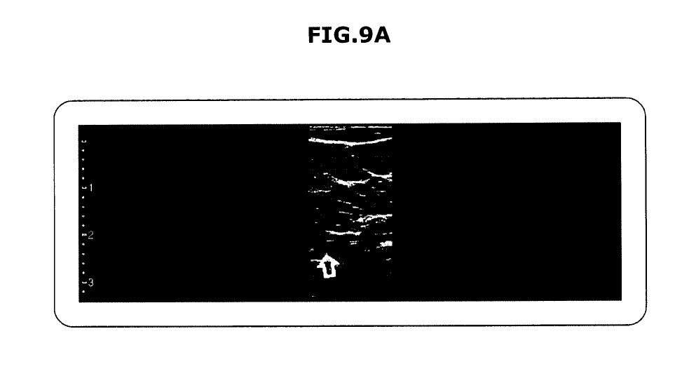

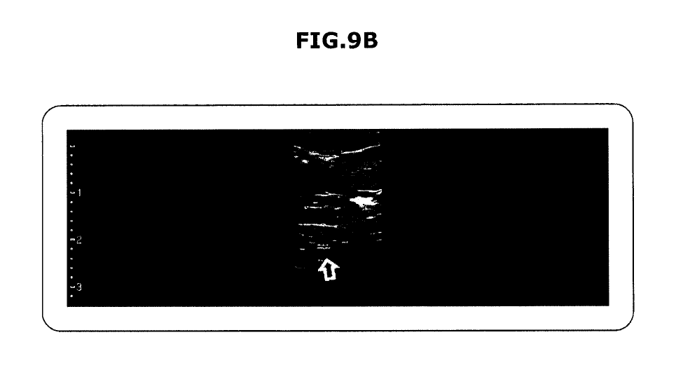

FIGS. 9A and 9B illustrate ultrasonic images of breasts taken using a probe with different frequencies.

Referring to FIG. 9A, FIG. 9A illustrates an ultrasonic image of breasts using a linear array probe using a high frequency. The probe using the high frequency is useful to obtain a clear image due to high resolution thereof. However, since high frequency ultrasonic waves have low penetration, it is difficult to obtain a clear image of a part deep inside an object. Accordingly, a probe using low frequency ultrasonic waves is necessary to obtain a relatively clear image of a place deep inside an object.

FIG. 9B illustrates an ultrasonic image of breasts taken using a linear array probe with a low frequency. Comparing FIGS. 9A and 9B with each other, even though an image obtained using a high frequency probe is clear in case of a region with a small depth in the image, an image obtained using a low frequency probe is more clear in case of a region with a great depth. Accordingly, the controller 500 may guide the user to change a probe based on a position of an object to be observed. As shown in FIGS. 9A and 9B, when to obtain an image of a region with a small depth, the user may be guided to change the probe with a high frequency probe. When to obtain an image of a region with a great depth, the user may be guided to change the probe with a low frequency probe.

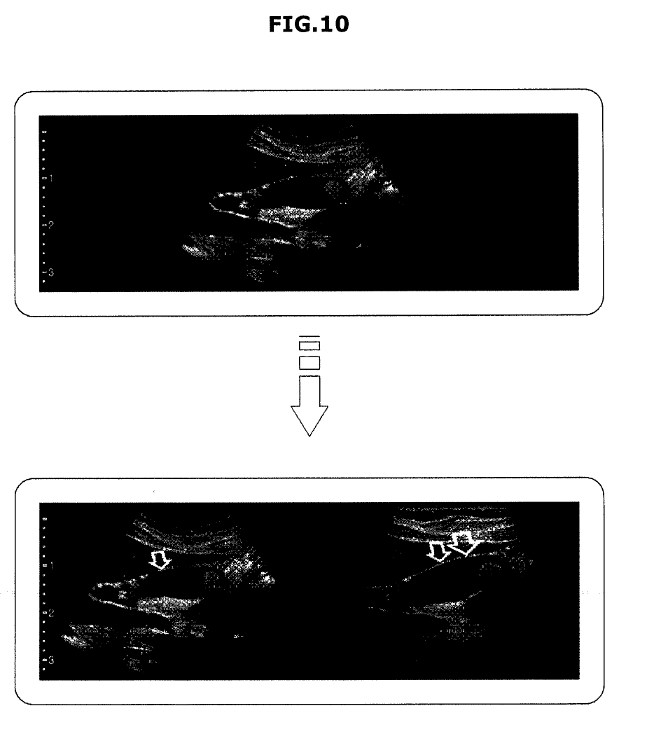

FIG. 10 illustrates an image of a gallbladder taken using different types of probes on one display according to one embodiment.

Referring to FIG. 10, FIG. 10 illustrates a process of replacing a probe with the linear array probe PL while taking an image of the gallbladder using the convex array probe PC based on the above description. A screen shown in FIG. 10 illustrates an image of the gallbladder taken using the convex array probe PC. The controller 500 may recommend replacing the existing convex array probe PC that is taking an image, with the linear array probe PL. When the user replaces the probe that is taking an image with the linear array probe PL, it is possible to show an image taken using the convex array probe PC and an image taken using the linear array probe PL at the same time. However, since it is difficult for the user to use the linear array probe PL that replaces the existing convex array probe PC at the same position, the controller 500 may show arrows for guiding to the same position on the display portion 550. In one embodiment of the present disclosure, the arrows may have different directions, sizes, and colors. Using the arrows, top and bottom positions and left and right positions may be changed. Also, with different colors of the arrows, a depth position of the probe may be guided to become the same as that of the convex array probe PC before replacing.

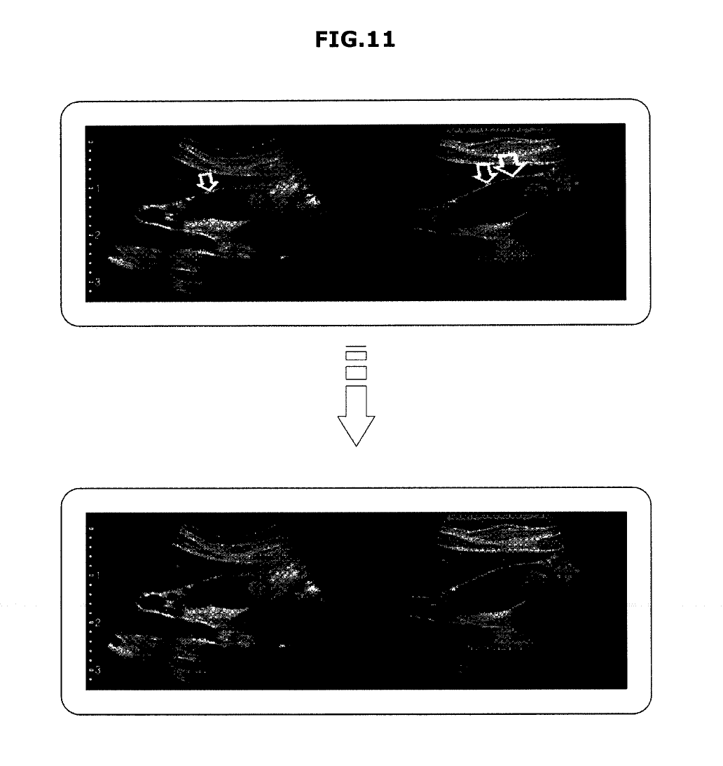

FIG. 11 illustrates adjusting positions of images of a gallbladder taken using different types of probes to the same position in accordance with one embodiment.

Referring to FIG. 11, FIG. 11 illustrates taking images of the gallbladder using the existing first ultrasound probe P1 and the second ultrasound probe P2 at the same position based on arrows shown in FIG. 11. The controller 500 may store a then image taken using the first ultrasound probe P1 and may display positions of images taken using the first ultrasound probe P1 and the second ultrasound probe P2 to allow the positions to become the same when the first ultrasound probe P1 is replaced with the second ultrasound probe P2. The user may make the positions of the first ultrasound probe P1 and the second ultrasound probe P2 to be the same based on the arrows shown in FIG. 10 and may display the images taken using the first and second ultrasound probes P1 and P2 on the display portion 550 as shown in FIG. 10.

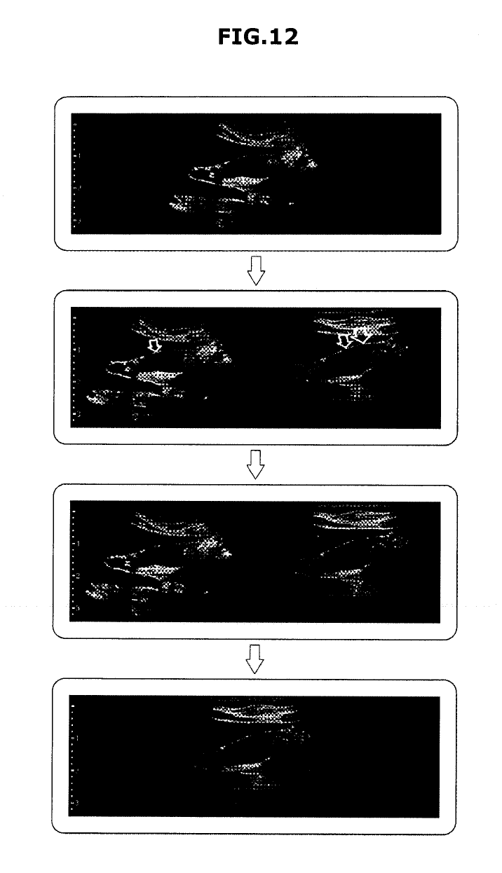

FIG. 12 illustrates changes in images shown on a display in accordance with one embodiment.

FIG. 12 illustrates overall operations in accordance with one embodiment of the present disclosure. Referring to FIG. 12, even though the user takes an image of a gallbladder using the first ultrasound probe P1, the controller 500 may recommend the second ultrasound probe P2 that is more adequate for taking an image of the gallbladder. Based the above description, the user may replace the first ultrasound probe P1 with the second ultrasound probe P2 and may adjust the first ultrasound probe P1 and the second ultrasound probe P2 to be positioned at the same place. When it is determined that the second ultrasound probe P2 is positioned at the same place as that of the first ultrasound probe P1, the controller 500 may display only an image taken by the second ultrasound probe P2 on the display portion 550.

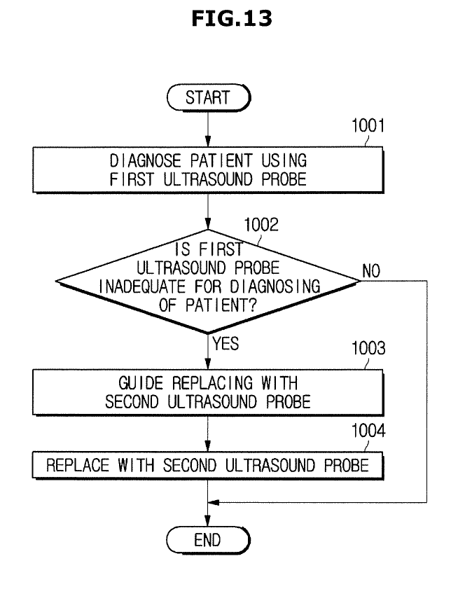

FIGS. 13 to 15 are flowcharts according to one embodiment.

Referring to FIG. 13, the user may diagnose a patient using the first ultrasound probe P1 (1001). The controller 500 may determine an organ of the patient to be diagnosed using the first ultrasound probe P1. As described above, the organ of the patient may be input by the user through input portion 540 and may be derived by the controller 500 using features of an image taken by the first ultrasound probe P1. When the first ultrasound probe P1 is determined to be inadequate for diagnosis after the user determines the organ of the patient to be diagnosed (1002), the controller 500 may guide the user to replace with the second ultrasound probe P2 (1003). When the user replaces the first ultrasound probe P1 with the second ultrasound probe P2, the controller 500 may guide the second ultrasound probe P2 to be positioned at the same position as that of the first ultrasound probe P1 using arrows on the display portion 550 (1003). Here, the controller 500 may display an image obtained by the first ultrasound probe P1 and an image obtained by the second ultrasound probe P2 at the same time for a smooth guide. When the user positions the second ultrasound probe P2 at the same place as that of the first ultrasound probe P1, the controller 500 may display only the image taken by the second ultrasound probe P2 (1003). Based on the above description, the user may replace the first ultrasound probe P1 with the second ultrasound probe P2 (1004).

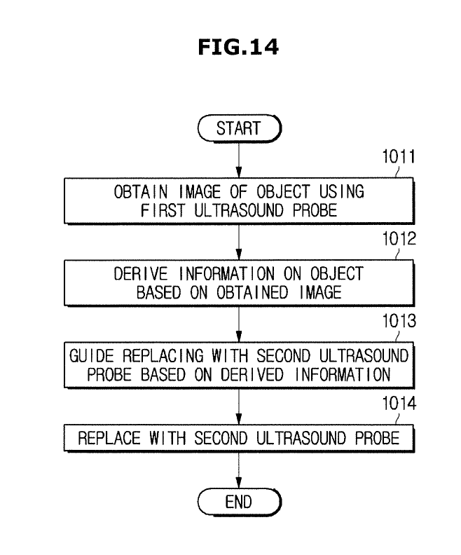

Referring to FIG. 14, the first ultrasound probe P1 may obtain an image of an object (1011) and the controller 500 may derive information on the object based on the obtained image (1012). The information on the object may include length, width, type, and disease information of the object. Based on the derived information, the controller 500 may guide the user to replace the first ultrasound probe P1 with the second ultrasound probe P2 (1013) and the user may replace the first ultrasound probe P1 with the second ultrasound probe P2 (1014).

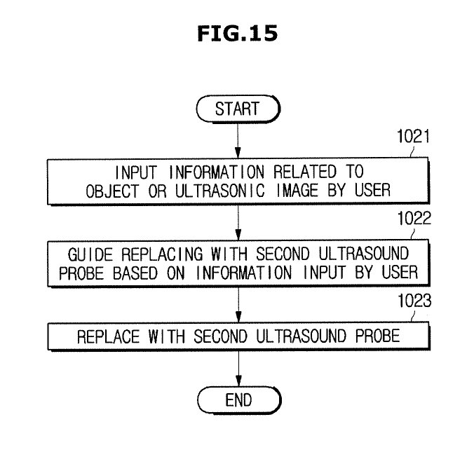

Referring to FIG. 15, the user may input information related to an object or information related to an ultrasonic image (1021). The information related to the object may include at least one of a length, a width, and a volume of the object, and the information related to the ultrasonic image may include at least one of an ROI of the object and depth information of the ultrasonic image input by the user. The controller 500 may guide the user to replace the first ultrasound probe P1 with the second ultrasound probe P2 based on the information input by the user (1022). The user may replace the first ultrasound probe P1 with the second ultrasound probe P2 according to the guide of the controller 500 (1023).

Meanwhile, the disclosed embodiments may be embodied as recording media that store commands executable by computers. The commands may be stored as the form of program codes and may generate program modules to perform operations of the disclosed embodiments when being executed by a processor. The recording media may be embodied as computer-readable recording media.

The computer-readable recording media may include all types of recording media that store computer-decodable commands. For example, there may be a read-only memory (ROM), a random-access memory (RAM), a magnetic tape, a magnetic disc, a flash memory, an optical data storage and the like.

As is apparent from the above description, an ultrasound imaging apparatus and a method of controlling the same in accordance with one embodiment of the present disclosure may recommend a probe adequate for an organ of a patient to be diagnosed to allow a user to replace an existing probe to efficiently and precisely diagnose the patient.

Although a few embodiments of the present disclosure have been shown and described, it would be appreciated by those skilled in the art that changes may be made in these embodiments without departing from the principles and spirit of the present disclosure, the scope of which is defined in the claims and their equivalents.

What is claimed is:

1. An ultrasound imaging apparatus comprising:
a first ultrasound probe configured to obtain an ultrasonic image of an object by transmitting and receiving ultrasonic signals;
a second ultrasound probe of a different type from the first ultrasound probe;
a display portion configured to display an ultrasonic image obtained by at least one of the first ultrasound probe and the second ultrasound probe; and
a controller configured to derive information of the object based on an ultrasonic image obtained by the first ultrasound probe and guide replacing the first ultrasound probe with the second ultrasound probe based on the information of the object.

2. The ultrasound imaging apparatus of claim 1, further comprising an input portion configured to receive information related to the ultrasonic image input from a user,
wherein the controller guides to replace the first ultrasound probe with the second ultrasound probe based on the information received by the input portion.

3. The ultrasound imaging apparatus of claim 1, further comprising an input portion configured to receive information of the object input from a user,
wherein the controller guides to replace the first ultrasound probe with the second ultrasound probe based on the information received by the input portion.

4. The ultrasound imaging apparatus of claim 1, wherein the controller stores an ultrasonic image obtained by the first ultrasound probe and displays the ultrasonic image obtained by the first ultrasound probe and an ultrasonic image obtained by the second ultrasound probe on the display portion when the first ultrasound probe is replaced with the second ultrasound probe.

5. The ultrasound imaging apparatus of claim 1, wherein the controller displays a moving image for guiding the second ultrasound probe to the same position as that of the first ultrasound probe on the display portion when the first ultrasound probe is replaced with the second ultrasound probe.

6. The ultrasound imaging apparatus of claim 1, wherein each of the first ultrasound probe and the second ultrasound probe is provided as any one of a convex array probe, a linear array probe, an endocavity array probe, and a phased array probe.

7. The ultrasound imaging apparatus of claim 1, wherein the second ultrasound probe obtains an ultrasonic image using ultrasonic waves at a different frequency band from that of the first ultrasound probe.

8. The ultrasound imaging apparatus of claim 6, wherein the controller guides replacing the first ultrasound probe with the second ultrasound probe that is a linear array probe when the object is determined as a gallbladder.

9. The ultrasound imaging apparatus of claim 6, wherein the controller guides replacing the first ultrasound probe with the second ultrasound probe that is a linear array probe when the object is determined as a liver.

10. The ultrasound imaging apparatus of claim 6, wherein the controller guides replacing the first ultrasound probe with the second ultrasound probe that is an endocavity array probe when the object is determined as a uterus.

11. The ultrasound imaging apparatus of claim 1, wherein the controller displays information of the second ultrasound probe on the display portion when it is guided to replace the first ultrasound probe with the second ultrasound probe.

12. A method of controlling an ultrasound imaging apparatus, comprising:
obtaining an ultrasonic image of an object by transmitting and receiving ultrasonic signals;
deriving information of the object based on an ultrasonic image obtained by a first ultrasound probe;
guiding replacing the first ultrasound probe with a second ultrasound probe based on the information of the object; and
displaying an ultrasonic image obtained by at least one of the first ultrasound probe and the second ultrasound probe.

13. The method of claim 12, wherein the guiding of replacing the first ultrasound probe with the second ultrasound probe comprises:
receiving information related to the ultrasonic image input from a user; and
guiding an input portion to replace the first ultrasound probe with the second ultrasound probe based on the information received at the input portion.

14. The method of claim 12, wherein the guiding of replacing the first ultrasound probe with the second ultrasound probe comprises:
receiving information of the object input from a user; and
guiding an input portion to replace the first ultrasound probe with the second ultrasound probe based on the information received at the input portion.

15. The method of claim 12, further comprising storing the ultrasonic image obtained by the first ultrasound probe,
wherein the displaying of the ultrasonic image comprises displaying the ultrasonic image obtained by the first ultrasound probe and an ultrasonic image obtained by the second ultrasound probe when the first ultrasound probe is replaced with the second ultrasound probe.

16. The method of claim 12, wherein the guiding of replacing the first ultrasound probe with the second ultrasound probe comprises displaying a moving image for guiding the second ultrasound probe to the same position as that of the first ultrasound probe.

17. The method of claim 12, wherein the guiding of replacing the first ultrasound probe with the second ultrasound probe comprises guiding replacing the first ultrasound probe with the second ultrasound probe that is a linear array probe when the object is determined as a gallbladder.

18. The method of claim 12, wherein the guiding of replacing the first ultrasound probe with the second ultrasound probe comprises guiding replacing the first ultrasound probe with the second ultrasound probe that is a linear array probe when the object is determined as a liver.

19. The method of claim 12, wherein the guiding of replacing the first ultrasound probe with the second ultrasound probe comprises guiding replacing the first ultrasound probe with the second ultrasound probe that is an endocavity array probe when the object is determined as a uterus.

20. The method of claim 11, wherein the guiding of replacing the first ultrasound probe with the second ultrasound probe comprises displaying information of the second ultrasound probe when it is guided that the first ultrasound probe is replaced with the second ultrasound probe.
